(19) United States
(12) Patent Application Publication
(10) Pub. No.: US 2022/0254459 A1
(43) Pub. Date: Aug. 11, 2022

(54) DATA PROCESSING METHOD, DATA PROCESSING DEVICE, COMPUTING DEVICE AND COMPUTER READABLE STORAGE MEDIUM

(71) Applicant: BOE TECHNOLOGY GROUP CO., LTD., Beijing (CN)

(72) Inventors: Jiao HUANG, Beijing (CN); Yanyang HU, Beijing (CN); Xiaoran SUN, Beijing (CN); Haiyan ZHAO, Beijing (CN)

(21) Appl. No.: 17/513,396

(22) Filed: Oct. 28, 2021

(30) Foreign Application Priority Data
Feb. 9, 2021 (CN) 202110178903.9

(51) Int. Cl. G16H 10/60; G16H 50/20

(52) U.S. Cl. CPC G16H 10/60; G16H 50/20

(57) ABSTRACT

A data processing method, a data processing device, a computing device, and a computer-readable storage medium are disclosed. The data processing method is carried out by a computing device, and the data processing method includes obtaining first health data, the first health data being marked as being associated with at least one user identifier, and obtaining second health data, the second health data including health data of a first user, and based on the first health data and the second health data, establishing an association relationship between the second health data and a target user identifier in the at least one user identifier, wherein the target user identifier is associated with the first user.

### CROSS REFERENCE

The present application claims the benefit of Chinese Patent Application for Invention No. 202110178903.9 filed on Feb. 9, 2021, the entire disclosure of which is incorporated herein by reference.

### TECHNICAL FIELD

The present disclosure generally relates to the field of data processing, and specifically relates to data processing methods, data processing devices, computing devices, and computer-readable storage media.

### BACKGROUND

With the development of information technology, sensor technology, Internet of Things (IoT) technology, and Internet technology, the collection of data and information has become more and more convenient, so the sources of data have become more multi-sourced. The Hospital Information System (HIS) may effectively collect, process and store various data generated by the user during the treatment in the hospital, so as to facilitate the grasp of the various health data of the user during the treatment in the hospital. The basic public health management system may summarize and process user data collected by medical institutions such as community central stations, township health centers, or village health centers to form user health profiles. The health monitoring system based on the IoT may collect and store health data in the user's daily life and health management process, which is convenient for long-term monitoring of the user's health status.

### SUMMARY

According to an aspect of the present disclosure, there is provided a data processing method, the data processing method being carried out by a computing device, the data processing method comprising: obtaining first health data, the first health data being marked as being associated with at least one user identifier; obtaining second health data, the second health data comprising health data of a first user; and based on the first health data and the second health data, establishing an association relationship between the second health data and a target user identifier in the at least one user identifier, wherein the target user identifier is associated with the first user.

In some embodiments, the second health data and the first health data come from different database systems.

In some embodiments, based on the first health data and the second health data, establishing an association relationship between the second health data and the target user identifier in the at least one user identifier comprises: determining whether there is the target user identifier in the at least one user identifier based on the first health data and the second health data; and in response to the presence of the target user identifier in the at least one user identifier, establishing an association relationship between the second health data and the target user identifier.

In some embodiments, after establishing the association relationship between the second health data and the target user identifier, further comprises: based on the first health data and the second health data, analysing the health status of the user associated with the target user identifier.

In some embodiments, the first health data comprises a plurality of first data indicating a first detection item, and each of the plurality of first data is related to one of the at least one user identifier, the second health data comprises second data indicating a first detection item, and based on the first health data and the second health data, establishing an association relationship between the second health data and the target user identifier in the at least one user identifier comprises: determining the similarity between the second data and the plurality of first data; based on the similarity between the second data and the plurality of first data, establishing an association relationship between the second health data and the target user identifier in the at least one user identifier.

In some embodiments, the second data has a data format different from the plurality of first data, and before determining whether there is the target user identifier in the at least one user identifier based on the first health data and the second health data, the data processing method further comprises: converting the format of the second data into the same format as the format of the plurality of the first data.

In some embodiments, the determining whether there is the target user identifier in the at least one user identifier based on the first health data and the second health data comprises: calculating the data volume of the health data corresponding to each of the at least one user identifier in the first health data; determining whether the data volume is greater than a first threshold; in response to the data volume being greater than the first threshold, determining whether there is the target user identifier in the at least one user identifier based on a content of the first health data and a content of the second health data; and in response to the data volume being not greater than the first threshold, determining whether there is the target user identifier in the at least one user identifier according to a preset rule.

In some embodiments, the determining the similarity between the second data and the plurality of first data comprises: respectively determining a distance value between the second data and each of the plurality of first data to obtain a plurality of distance values; selecting a first set from the plurality of distance values, wherein the first set comprises at least one distance value that meets a predetermined filtering condition; for each of the at least one user identifier, respectively determining the number of distance values in the first set and associated with each user identifier, wherein, based on the similarity between the second data and the plurality of first data, establishing an association relationship between the second health data and the target user identifier in the at least one user identifier comprises: determining the target user identifier based on the number of distance values associated with each user identifier; and based on the target user identifier, establishing an association relationship between the second health data and the target user identifier in the at least one user identifier.

In some embodiments, the determining the target user identifier based on the number of distance values associated with each user identifier comprises: determining the user identifier associated with the largest number of distance values in the first set as the target user identifier.

In some embodiments, the determining the target user identifier based on the number of distance values associated with each user identifier comprises: calculating the ratio of the maximum number of distance values in the first set and associated with each user identifier to the sum of the number of distance values in the first set and associated with each user identifier; determining whether the ratio is greater than a second threshold; and in response to the ratio being greater than the second threshold, determining the user identifier associated with the largest distance values in the first set as the target user identifier.

In some embodiments, the first health data comprises a plurality of first data indicating a first detection item, and each of the plurality of first data is related to one of the at least one user identifier, the second health data comprises second data indicating a first detection item, and based on the first health data and the second health data, establishing an association relationship between the second health data and the target user identifier in the at least one user identifier comprises: obtaining a prediction result based on the second data and the first prediction model, and the first prediction model is trained based on the association relationship between each of the plurality of first data and the at least one user identifier; and establishing an association relationship between the second health data and the target user identifier in the at least one user identifier based on the prediction result.

In some embodiments, based on the first health data and the second health data, analysing the health status of the user associated with the target user identifier comprises: determining a collection moment corresponding to the first health data and a collection moment corresponding to the second health data; arranging the first health data and the second health data in chronological order to obtain a health data sequence; and based on the health data sequence, analysing the health status of the user associated with the target user identifier.

In some embodiments, based on the health data sequence, analysing the health status of the user associated with the target user identifier comprises: obtaining a user feature sequence associated with the target user identifier; based on the health data sequence, the user feature sequence, and a second prediction model, obtaining an analysis result of the user's health status associated with the target user identifier, wherein the second prediction model is trained based on the user's historical health data sequence, historical user feature sequence and historical health status.

In some embodiments, the first prediction model is a neural network model.

In some embodiments, the second prediction model is at least one of an ARIMA model, a neural network model, or a Prophet model.

In some embodiments, wherein the obtaining a prediction result based on the second data and the first prediction model comprises: combining the second data with data of other dimensions associated with the second data to form a data sample; normalizing the data sample; and inputting the normalized data sample the first prediction model to obtain a prediction result.

According to another aspect of the present disclosure, there is provided a data processing device, comprising: a first obtainer configured to obtain first health data, the first health data being marked as being associated with at least one user identifier; a second obtainer configured to obtain second health data, the second health data comprising health data of a first user; and an establisher configured to establish an association relationship between the second health data and a target user identifier in the at least one user identifier based on the first health data and the second health data, wherein the target user identifier is associated with the first user.

According to a further aspect of the present disclosure, there is provided a computing device, the computing device comprising a memory, a processor, and computer instructions stored in the memory and executable on the processor, the processor being configured to implement the data processing method as described above when the computer instruction is executed.

In some embodiments, the memory comprises an IoT data lake.

According to a further aspect of the present disclosure, there is provided a non-transitory computer-readable storage medium having computer instructions stored thereon, the computer instructions being configured to implement the data processing method as described above.

### BRIEF DESCRIPTION OF DRAWINGS

By reading the detailed description of the non-limiting embodiments with reference to the following drawings, other features, purposes and advantages of the present disclosure will become more apparent:

FIG. 1 is an architecture diagram of an implementation environment of a data processing method provided by some embodiments of the present disclosure;

FIG. 2 is a schematic structural diagram of a multi-source health data intelligent archiving system provided by some embodiments of the present disclosure;

FIG. 3 is a schematic diagram of a process of establishing a health profile provided by some embodiments of the present disclosure;

FIG. 4 is a schematic flowchart of a data processing method provided by some embodiments of the present disclosure;

FIG. 5 is a schematic flowchart of another data processing method provided by some embodiments of the present disclosure;

FIG. 6 is a schematic diagram of a display interface of an application program according to some embodiments of the present disclosure;

FIG. 7 is a schematic diagram of another display interface of an application program according to some embodiments of the present disclosure;

FIG. 8 shows a schematic diagram of second data and a plurality of first data according to an embodiment of the present disclosure;

FIG. 9 shows at least part of the sub-steps of a data processing method according to some embodiments of the present disclosure;

FIG. 10a is a schematic diagram of a process of obtaining a prediction result based on a multi-layer classification neural network according to some embodiments of the present disclosure;

FIG. 10b is a schematic diagram of a process of obtaining a prediction result based on a single-class neural network according to some embodiments of the present disclosure;

FIG. 11 is a schematic structural diagram of a data processing device provided by some embodiments of the present disclosure; and

FIG. 12 is a schematic structural diagram of a computing device provided by some embodiments of the disclosure.

### DETAILED DESCRIPTION

The present disclosure will be further described in detail below with reference to the accompanying drawings and embodiments. It may be understood that the specific embodiments described here are only used to explain the related invention, but not to limit the invention. In addition, it should be noted that, for ease of description, only the parts related to the invention are shown in the drawings.

It should be noted that the embodiments in the present disclosure and the features in the embodiments may be combined with each other without conflicting. Hereinafter, the present disclosure will be described in detail with reference to the drawings and in conjunction with the embodiments.

The terms used in the present disclosure are only used to describe each exemplary embodiment in the present disclosure, and are not intended to limit the present disclosure. As used herein, the singular forms “a,” “an,” and “the” are intended to also comprise the plural forms, unless the context clearly dictates otherwise. It should also be understood that the terms “comprising” and “comprising” when used in the present disclosure refer to the existence of the mentioned features, but do not exclude the existence of one or more other features or the addition of one or more other features. As used herein, the term “and/or” comprises any and all combinations of one or more of the associated listed items. It will be understood that although the terms “first”, “second”, “third”, etc. may be used herein to describe various features, these features should not be limited by these terms. These terms are only used to distinguish one feature from another.

Unless otherwise defined, all terms (comprising technical and scientific terms) used in the present disclosure have the same meanings as commonly understood by those of ordinary skill in the art to which the present disclosure belongs. It should also be understood that terms such as those defined in commonly used dictionaries should be interpreted as having meanings consistent with their meanings in the relevant field and/or the context of this specification, and will not be interpreted in an idealized or overly formal sense, unless explicitly defined as such herein.

For the health data collected by the user in the daily life, there may be an archiving error and abnormal health data may be archived, resulting in poor health data quality of the user. In addition, the Hospital Information System, the basic public health management system, and the health monitoring system based on the IoT are usually systems that operate independently. The data of these 3 systems each form a system. The user health status obtained based on user data of different systems may be different, which cannot truly reflect the real health status of the user, and it is not conducive to the user's health management and disease diagnosis. Health data is sometimes archived abnormally and the health data is single. For example, for residents, it is impossible to form a multi-source unified and complete health profile, and for chronic disease management, there is no effective combination of pre-hospital, in-hospital and post-hospital. In terms of home health/chronic disease data collection, there are conditions that are not measured by the person actually using the equipment or abnormal data collected by abnormal operations. In addition, the elderly who do not understand the operation of smart mobile terminals and the abnormal data that are not easy to actively delete may easily cause data confusion, poor data quality, and are not conducive to the formation of a complete personal health profile. In this way, it is easy to cause institutions at all levels to be unable to effectively screen residents for chronic diseases such as hypertension, diabetes, and hyperlipidaemia, create profiles, and realize dynamic hierarchical management and health education.

In view of the aforementioned shortcomings or deficiencies in the prior art, it is desirable to provide a data processing method, data processing device, computing device, and computer-readable storage medium that improve the quality of user health data and may truly reflect the user's health status.

FIG. 1 schematically shows an architecture diagram of an implementation environment of a data processing method according to an embodiment of the present disclosure. As shown in FIG. 1, the implementation environment architecture comprises: a first computing device 110, a second computing device 112, a first terminal 1101, a third computing device 120, and a second terminal 130. The third computing device 120 establishes a network connection with the first computing device 110, the second computing device 112, and the second terminal 130. The first terminal 1101 establishes a direct network connection with the second computing device 112 and then indirectly connects to the third computing device 120 via the network. The implementation environment architecture also comprises: electronic health monitoring equipment 204, which may be configured to monitor and collect user health data. The electronic health monitoring equipment 204 may establish a direct network connection with the third computing device 120, or establish a direct network connection with the second terminal 130 and then indirectly connect to the third computing device 120 via the network.

For example, the first computing device 110, the second computing device 112, and the third computing device 120 may be computers, servers, or server clusters with data processing capabilities. For example, the first terminal 1101 may be a smart electronic health monitoring device (such as a blood pressure meter, a blood sugar meter, and a wearable smart monitoring device), and the second terminal 130 may be an electronic device such as a mobile phone, a wearable device, a tablet computer, and a personal computer.

In some embodiments, the first database system (not shown) may run on the first computing device 110, and the second database system (not shown) may run on the second computing device 112. The third computing device 120 may be configured to run a multi-source health data intelligent archiving system (not shown), and the multi-source health data intelligent archiving system may comprise a third database system (not shown). For example, the third computing device 120 may be configured to carry out the data processing method provided by the present disclosure (see the description below).

In some embodiments, the second terminal 130 may measure or collect the initial health data of at least one user. For example, the second terminal 130 may bind the device identifier of the second terminal 130 to the at least one user identifier in response to a binding operation on the at least one user identifier. The second terminal 130 may send the bound device identifier and at least one user identifier to the third computing device 120 running a third database system (for example, an IoT health monitoring system). The third database system may store the bound device identifier and at least one user identifier in the database.

After the initial health data is measured or collected by the second terminal 130, the initial health data, device identifier, and user identifier may be bound, and the bound initial health data, device identifier, and user identifier may be sent together to the third computing device 120 running the third database system. The multi-source health data intelligent archiving system 200 (see below) associates the initial health data with the user identifier. In this way, the binding and association of initial health data, device identifier, and user identifier are realized. For example, the user identifier may be a user identifier number or a user health insurance number.

In some embodiments, the user may register a user account through the application (or WeChat mini program, Alipay mini program, and/or web management end, etc.) associated with the multi-source health data intelligent archiving system in the second terminal 130, and the second terminal 130 obtain the user's basic information (for example, at least one user's user ID) and synchronize it to the third computing device 120 for storage in response to the user's information selection or filling operation.

In the embodiments of the present disclosure, the basic user information may be basic information of the user corresponding to the user identifier and information such as the user's living habits. The basic information may be, for example, information such as height, age, and gender, and the user's living habits may be, for example, information such as whether smoking, whether drinking, living environment, labor intensity, and exercise habits.

For example, when storing basic user information, the data of basic user information may be: for gender, male may be represented by number 1, and female may be represented by number 0; for smoking habits, smoking may be represented by number 1, and no smoking may be represented by the number 0; for the drinking habit, drinking may be represented by the number 1, and not drinking may be represented by the number 0; for the living environment, southern cities may be represented by number 0, southern rural areas may be represented by number 1, northern cities may be represented by number 2, and northern rural areas may be represented by the number 3; for labor intensity, bed rest may be represented by the number 0, light physical labor may be represented by the number 1, medium physical labor may be represented by the number 2, and heavy physical labor may be represented by the number 3; for exercise habits, substantially not exercising may be represented by number 0, exercising once a week on average may be represented by the number 1, exercising twice a week on average may be represented by the number 2, and exercising 3 times a week on average may be represented by the number 3.

In some embodiments, the first database system may be a Hospital Information System, and the first computing device 110 running the first database system may collect and store various secondary health data (for example, drug prescription data) generated by users during medical testing in hospitals, community centers, township health centers, or village health centers, and the second health data is sent to the database in the third computing device 120 for storage.

In some embodiments, the second database system may be a basic public health management system. The second computing device 112 running the second database system may establish a network connection with the first terminal 1101 used by the public health center to obtain and store the second health data detected by the user using the first terminal 1101. For example, the second computing device 112 may send the second health data to a database in the third computing device 120 via the network for storage.

For example, the database in the third computing device 120 may be implemented as an IoT data lake, which has the following advantages and effects: 1. Compatible with the IoT technology: supports multi-network and multi-protocol device access, comprising Wi-Fi, Wi-Fi+BLE, BLE, BLE-Mesh, Zigbee, 3/4G, NB-IoT, etc.; 2. Access multiplexing: Port hardware access once develops multiple system multiplexing, and data management once develops multiple system multiplexing, avoiding repeated development of port access; 3. Distributed development framework: uses a distributed file system to store data, with high scalability; the use of open source technology also reduces storage costs and has higher flexibility; 4. Multi-dimensional data processing engine: provides data penetration capabilities for various business systems, facilitates unified management of data access, storage, conversion, and distribution of business systems, supports structured & unstructured data processing, and may view global data & complete process analysis in real time; 5. Rule engine technology: According to business scale requirements, rules may be expanded online quickly without business affecting each other; 6. Security encryption technology: for different security levels, the platform provides multiple security authentication methods, providing multiple protections to ensure the safety of equipment.

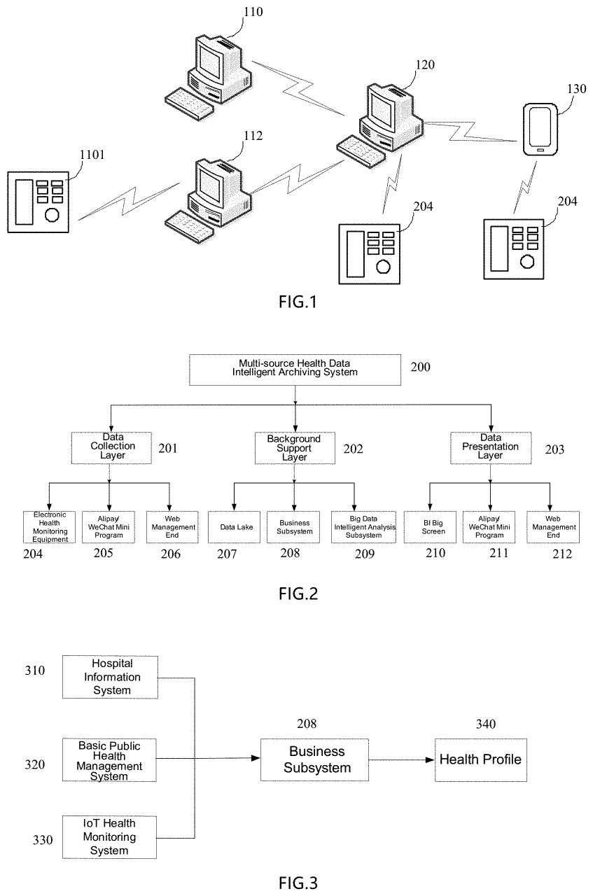

As shown in FIG. 2, the multi-source health data intelligent archiving system 200 may comprise a data collection layer 201, a background support layer 202, and a data presentation layer 203.

For example, the data collection layer 201 may be configured to obtain user information, device information, and user health data of the user on the Alipay/WeChat mini program 205 and/or web management end 206 of the second terminal 130.

In some embodiments, the background support layer 202 may be configured to save the data collected by the data collection layer 201 to the data lake 207. The background support layer 202 may also be configured to interface with the first database system and the second database system to obtain the second health data detected by the first terminal 1101 sent by the second computing device 112 or the second health data sent by the first computing device 110. The background support layer 202 may also be configured to use the business subsystem 208 and the big data intelligent analysis subsystem 209 to process the data in the data lake 207 to obtain data processing results. For example, data lake 207 may be configured to support desensitization management of hardware collection equipment data; business subsystem 208 may be configured for unified management of users and data, organizational grid management, multi-source data integration, intelligent archiving, etc.; big data intelligent analysis subsystem 209 may be configured to perform data pre-processing, user data trend analysis, data similarity calculation, and the like.

Business subsystem 208 supports for interfacing with HIS system 310 and basic public health management system 320 to obtain the second health data from different database systems. It should be understood that in the description herein, the expression “health data comes from different database systems” or “health data from different database systems” means that the terminal devices that collect these health data belong to different database systems.

To ensure user privacy, data may be transmitted over the network by encrypting the user identifier field. In addition, the business subsystem 208 may be configured to use the data processing method provided by the embodiments of the present disclosure (see the description below) to associate the health data of different sources in the data lake 207 with the user identifier according to the associated information of the managed device and the user, integrating data from different equipment sources in different scenarios. Therefore, the user health data from different database systems is integrated through the data processing method provided by the embodiments of the present disclosure.

For example, the data presentation layer 203 may be configured to send the data processing result (for example, a health analysis report) to the Alipay/WeChat mini program 211 or the web management end 212 of the second terminal 130 for display. Alternatively, the data presentation layer 203 may be configured to send the data processing results to the business intelligence (BI) big screen 210 of hospitals, community central stations, township health centers, or village health centers for display, which is convenient for users and doctors to obtain the user's health data in time.

In some embodiments, for each user identifier in the multi-source health data intelligent archiving system 200 running on the third computing device 120, as shown in FIG. 3, the business subsystem 208 may obtained the second health data collected by the first database system in the data lake 207, the second health data collected by the second database system, and the initial health data collected by the third database system. The initial health data may be marked as associated with at least one user identifier by the multi-source health data intelligent archiving system 200 to form first health data. The business subsystem 208 may use the data processing method provided by the embodiments of the present disclosure to integrate the first health data and the second health data to obtain the integrated health data, and store the integrated health data in the user profile corresponding to each user identifier, thereby establishing user security health profile 340. For example, the first database system may be hospital information system 310, the second database system may be basic public health management system 320, and the third database system may be IoT health monitoring system 330.

The big data intelligent analysis subsystem 209 may obtain the health status analysis result corresponding to each user identifier based on the health profile, and the user may use the application program associated with the multi-source health data intelligent archiving system 200 in the second terminal 130 to obtain the health status analysis results. Alternatively, when the server 120 determines that the health status analysis result corresponding to the user identifier is abnormal, the health status analysis result may be sent to the second terminal 130, so that the user may obtain the abnormal user health status information in time. The abnormal health status analysis result is sent to the second computing device 112 running the second database system, which is convenient for doctors or health managers to return visits to patients in time. In some embodiments, in order to protect user privacy and data security, data between systems and devices is encrypted data during transmission.

In the description of the present disclosure, the term “first health data” refers to health data as follows: the health data whose the initial health data has been associated with the user identifier by the multi-source health data intelligent archiving system 200 (i.e., the first health data is marked as associated with at least one user identifier by the multi-source health data intelligent archiving system 200), such as health data that has been archived in the profile directory of the user identifier; the term “second health data” is other health data different from the first health data. For example, the second health data may be health data sent by the first computing device 110 to the third computing device 120 or health data sent by the second computing device 112 to the third computing device 120. In some cases, the health data collected by the second terminal 130 or electronic health monitoring equipment 204 and sent to the third computing device 120 is only bound to the device identifier of the second terminal 130 and not to any user identifier, or is bound to the user identifier but not yet associated with the user identifier by the multi-source health data intelligent archiving system 200, such health data also belongs to the second health data. In related technologies, for the second health data obtained by the third computing device 120, archiving errors and abnormal health data may be archived. For example, there are three users A1, A2, and A3 for the same blood pressure meter. Assuming that user A1 uses the blood pressure meter to perform a blood pressure measurement to obtain blood pressure health data, the third computing device 120 may archive the blood pressure health data in the health profile of user A2 or user A3, causing the user's health data to be abnormal; and, suppose that user B1 uses the blood pressure meter to take a blood pressure measurement to obtain blood pressure health data, the third computing device 120 will generally archive the blood pressure health data in the health data table of user A1, user A2, or user A3, causing the user's health data to be abnormal.

In the embodiment of the present disclosure, the third computing device 120 may obtain the first health data from the data lake 207, and the first health data comprises health data associated with at least one user identifier. The third computing device 120 may obtain second health data, the second health data comprises the health data of the first user. The third computing device 120 may establish an association relationship between the second health data and the target user identifier in the at least one user identifier based on the first health data and the second health data, wherein the target user identifier is associated with the first user. In this way, determining the association relationship between the second health data and the target identifier user may prevent incorrect archiving of the second health data. When the target user identifier does not exist in at least one user identifier, it may be determined that the second health data is abnormal health data, and the abnormal health data may be deleted or re-archived, without archiving the abnormal second health data to an existing user identifier.

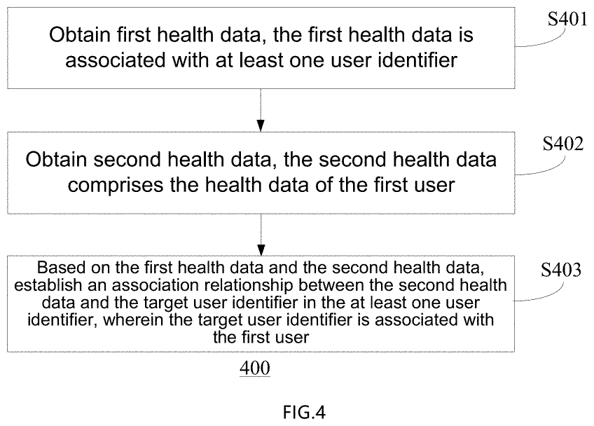

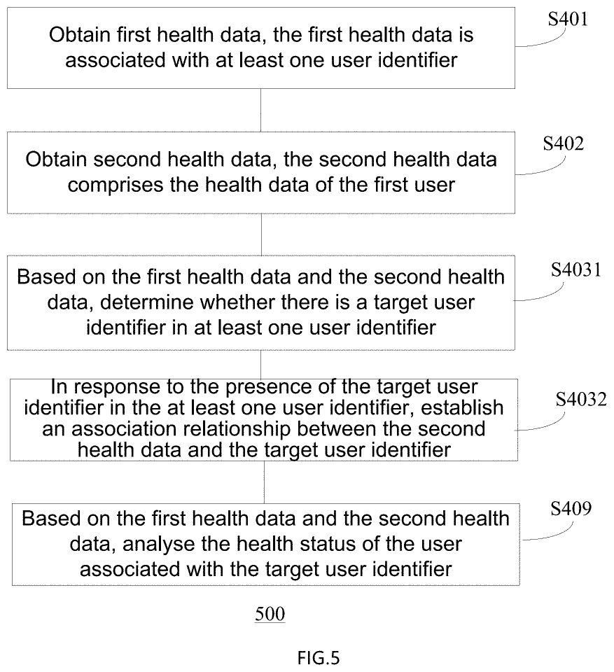

The data processing method 400 according to an embodiment of the present disclosure will be specifically described below in conjunction with FIG. 4. For example, the data processing method 400 may be carried out by the third computing device 120 shown in FIG. 1. As shown in FIG. 4, the data processing method 400 may comprise the following steps:

S401, obtain first health data, the first health data is marked as being associated with at least one user identifier.

For example, the data collection layer 201, the data lake 207, and the business subsystem 208 may form a third database system (for example, an IoT health monitoring system). For example, the multi-source health data intelligent archiving system 200 may comprise a third database system (for example, an IoT health monitoring system). With the help of the IoT health monitoring system, the data collection layer 201 may be configured to directly obtain user information, equipment information, and user initial health data collected by the electronic health monitoring equipment 204 connected to the IoT health monitoring system network. The multi-source health data intelligent archiving system 200 marks the user's initial health data as being associated with the user identifier, thereby forming the first health data and storing the formed first health data in the data lake 207. The first health data is marked as associated with at least one user identifier, and the at least one user identifier may be a user identifier bound to the electronic health monitoring equipment 204 or the second terminal 130 used to generate the initial health data. For example, if the user registers and logs in to perform the application in the second terminal 130 and then remotely controls the electronic health monitoring equipment 204 for physical examination, the collected initial health data may be bound to the user's registered account (the user's identity information may be associated), a device identifier of the second terminal 130 and the device identifier of electronic health monitoring equipment 204, and uploaded to the third computing device 120 therewith. The multi-source health data intelligent archiving system 200 associates these initial health data with the user identifier, thereby the first health data is formed and the formed first health data is stored in the data lake 207.

After forming the first health data, the multi-source health data intelligent archiving system 200 may obtain the first health data from the data lake 207.

For example, the IoT health monitoring system 330 may collect the initial health data of the first health data based on the electronic health monitoring equipment 204 such as the IoT blood pressure meter and the IoT blood sugar meter in the home scene, and then the initial health data is real-time upload to data lake 207 based on mobiles network such as 2G, 3G, 4G, etc. For example, the IoT health monitoring system 330 may collect the initial health data of the first health data based on the electronic health monitoring equipment 204 such as the bone density analyser or the body composition analyser of the health cabin, and then the initial health data is collected in the cabin workstation, and uploaded to data lake 207 in real time via the network based on the port technology. For example, the IoT health monitoring system 330 may collect and record the user's measurement time, measurement results, and basic user information such as age, gender, height, weight, and lifestyle habits history of illness, history of current illness, history of allergies, etc. based on WeChat/Alipay mini program 205, web management end 206, etc. on the second terminal 130, and collect this information in the business subsystem 208 for management. For example, the data lake 207 may be configured to receive the initial health data collected by the electronic health detection device 204 to realize unified storage and management of health data.

S402, obtain second health data, the second health data comprises the health data of the first user.

In some embodiments, for example, the second health data may be at least one of health data sent by the first computing device 110 to the third computing device 120, health data sent by the second computing device 112 to the third computing device 120, or the newly collected health data by the third computing device 120 through the IoT health monitoring system 330 (not yet associated with the user identifier by the multi-source health data intelligent archiving system 200). The background support layer 202 may be configured to interface with the first database system and the second database system to obtain the second health data sent by the first computing device 110 or the second computing device 112. The second health data comprises health data of the first user. For example, the multi-source health data intelligent archiving system 200 may regularly send data acquisition requests to the first computing device 110 or the second computing device 112, and the first computing device 110 or the second computing device 112 regularly sends the updated data in the first database system or the second database system to the third computing device 120 in response.

S403: Based on the first health data and the second health data, establish an association relationship between the second health data and the target user identifier in the at least one user identifier, wherein the target user identifier is associated with the first user.

The third computing device 120 may establish an association relationship between the second health data and the target user identifier in the at least one user identifier based on the first health data and the second health data, where the target user identifier is associated with the first user.

For example, the first health data is marked as being associated with multiple user identities (hereinafter referred to as “first user identifier”), and the second health data may also comprise the user identifier of the first user (hereinafter referred to as “second user identifier”). In this case, the second user identifier is matched with multiple first user identifiers, and if the matching is successful, the first user identifier that is successfully matched is used as the target user identifier, and association relationship between the second health data and the target user identifier is established. In this way, the integration of the first health data and the second health data associated with the same user identifier is completed, and the health data from multiple sources are integrated to form a health profile based on the multi-source health data. It helps to form a more authentic and effective personal health data profile, and at the same time helps to promote “integrated” health management services. The multi-source health data intelligent archiving system 200 covers multiple scenarios and forms a complete health management system, which is helpful for the promotion and development of medical community and medical consortium. In addition, it provides convenience for the following use of health profiles to analyse the health of the same user.

In some embodiments, the second health data and the first health data come from different database systems respectively. As described above, in the description of this article, the expression “the second health data and the first health data come from different database systems” means that the terminal devices that collect the second health data or the first health data belong to different database systems. For example, the terminal device that collects the first health data belongs to the IoT health monitoring system 330, and the terminal device that collects the second health data belongs to the hospital information system 310 or the basic public health system 320. For example, the second health data from the basic public health system 320 may be the diagnostic room, the clinic, manual blood pressure and blood sugar monitoring data; the first health data from the IoT health monitoring system 330 may be the blood pressure and blood sugar monitoring data from the IoT blood pressure meter and blood sugar meter; the second health data from the hospital information system 310 may be the medication data prescribed to the patient. These data from different sources may use an ID number as a user identifier, for example, and the user identifier is bound to it. In this way, an association may be established between the same user identifier and health data from different database systems.

In some embodiments, as shown in FIG. 5, step S403 comprises the following steps: S4031, based on the first health data and the second health data, determine whether there is a target user identifier in at least one user identifier; and S4032, in response to there being a target user identifier in the at least one user identifier, an association relationship between the second health data and the target user identifier is established. In this way, in response to there being a target user identifier in at least one user identifier, the association relationship between the second health data and the target user identifier is established; and in response to there not being a target user identifier in at least one user identifier (that is, the first user identifier is not associated with the first user, that is, the first health data is not associated with the first user), a manual review may be prompted or a new user profile corresponding to the first user may be created in the multi-source health data intelligent archiving system 200.

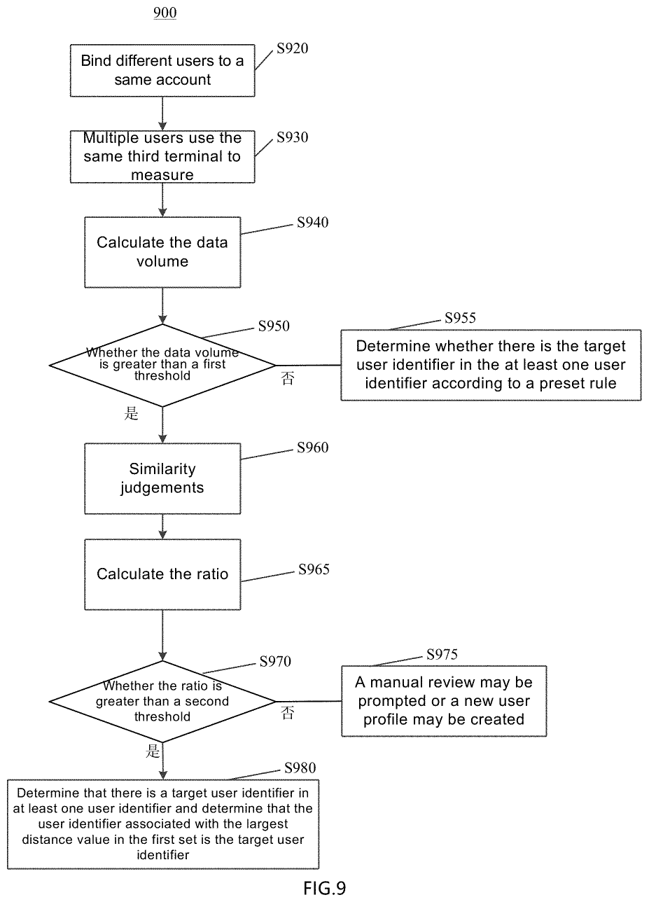

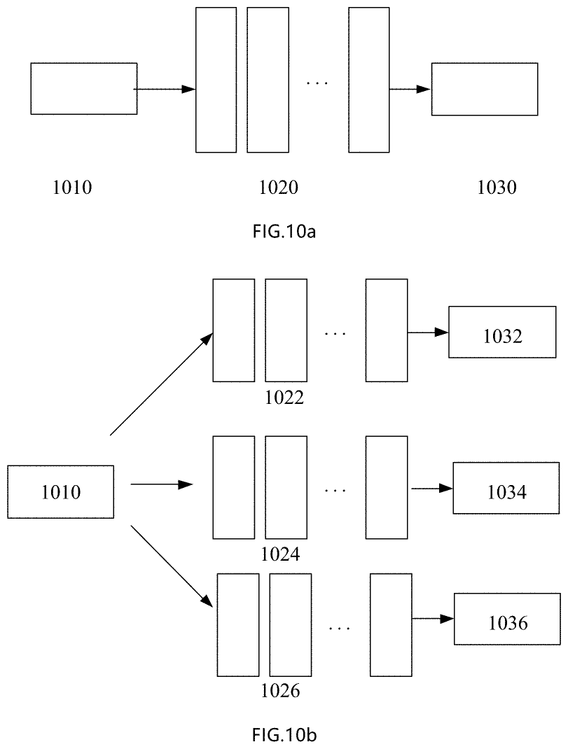

In some embodiments, Step S4031 comprises: calculating the data volume of the health data corresponding to each of the at least one user identifier in the first health data; determining whether the data volume is greater than a first threshold. In response to the data volume being greater than the first threshold, based on the content of the first health data and the content of the second health data, it is determined whether there is a target user identifier in the at least one user identifier (for example, see the description of FIGS. 9 and 10b below). In response to the data volume not being greater than the first threshold, it is determined whether there is the target user identifier in the at least one user identifier according to a preset rule. When the data volume is not greater than the first threshold, it is determined that the data volume does not meet the conditions for automatic archiving of the second health data, and the second health data may be processed according to preset rules to determine whether there is a target user in at least one user identifier. The preset rule may be, for example, that the staff compares the second health data with the first health data to determine the target user identifier corresponding to the second health data; the user manually determines the target user identifier corresponding to the second health data; or determines that there is no target user identifier in at least one user identifier and the second health data is abnormal health data, and a new profile of abnormal health data is established or the abnormal health data is eliminated. The first threshold of the data volume may be determined based on actual needs, which is not limited in the embodiment of the present application.

Since the second health data is automatically archived after determining that the data volume is greater than the first threshold, it may ensure that the data processing method is highly sensitive to abnormal second health data, and ensure the accuracy of the result of automatic archiving of the second health data.

It should be noted that, in the embodiments of this application, as the running time of the multi-source health data intelligent archiving system increases, the amount of sample data in the sample data set corresponding to each user identifier will gradually increase, and in the process of automatic archiving of the second health data, there are more and more historical health data (which may serve as the first health data) corresponding to the user identifier. If all the first health data is used as a sample detection data set, it will increase the consumption of computing resources as well as time, the newly obtained preset number of first health data may be used as the sample data set, where the preset number may be determined based on actual needs, which is not limited in the embodiment of the present application.

In some embodiments, after step S4032, the data processing method 400 further comprises: S409, based on the first health data and the second health data, analysing the health status of the user (for example, the first user) associated with the target user identifier.

In some embodiments, step S409 comprises: determining the collection moment corresponding to the first health data and the collection moment corresponding to the second health data; arranging the first health data and the second health data in chronological order to obtain a health data sequence; and based on the health data sequence, analysing the health status of the user associated with the target user identifier.

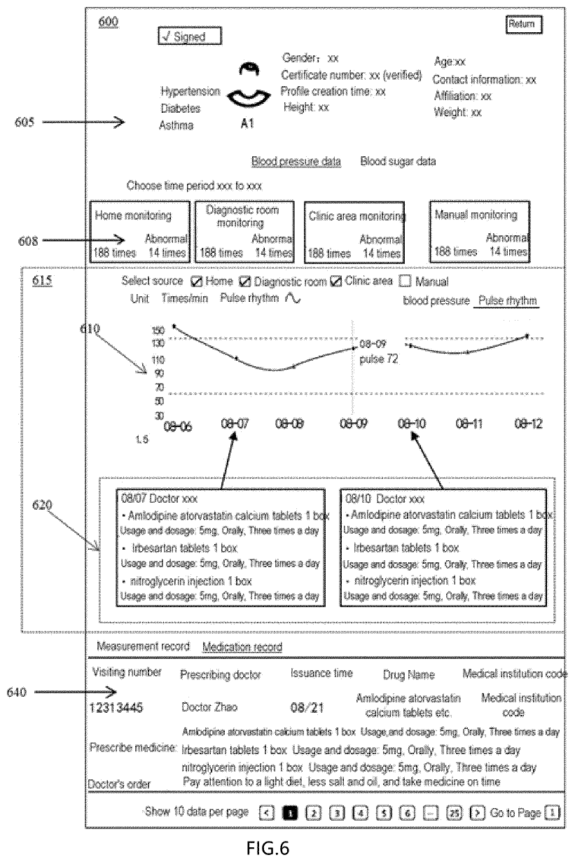

For example, as shown in FIG. 6, for the user A1, the third computing device 120 may determine the collection moment corresponding to the first health data (pulse value data 610) and the collection moment corresponding to the second health data (drug information data 620). As shown in FIG. 6, the pulse value data and the medication information data are arranged in chronological order, and a health data sequence 615 arranged in chronological order is obtained. For example, the first health data may also comprise various health data such as blood pressure value and blood sugar value. Based on the health data sequence 615, the health status of the user associated with the target user identifier may be analysed. For example, the user may intuitively understand the trend of the pulse value data 610 over time, and then analyse the health status.

In some embodiments, as shown in FIG. 6, the user may select the source of the data in the health data sequence 615 on the application interface of the second terminal 130. For example, the user may choose to display only different options such as “home”, “diagnostic room”, “clinic area”, “manual”, corresponding to different monitoring time or different monitoring conditions respectively.

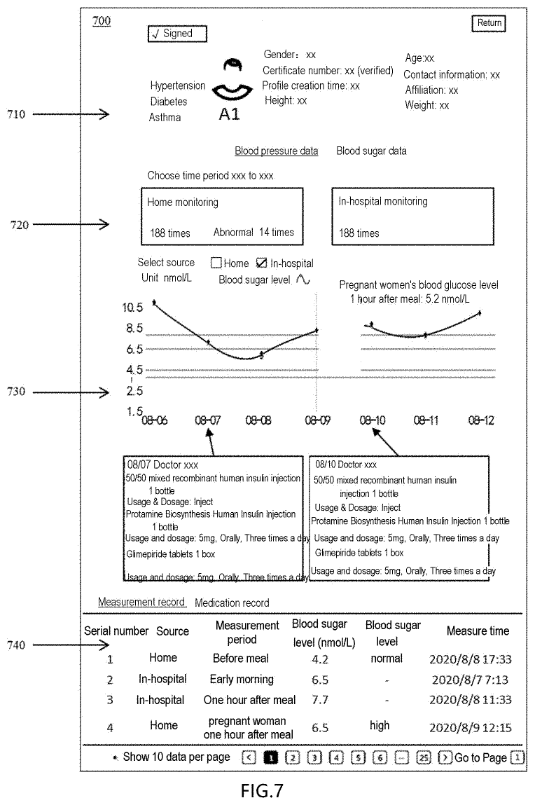

During the display of the user's health status analysis result, the application display interface of the second terminal 130 may be as shown in FIGS. 6-7. The content displayed on the display interface may comprise: user basic information, test items, health data sources and times; health data graphs, measurement records, and medication records, etc.

As shown in FIG. 6, the display interface 600 comprises: the user's basic information displayed in the first display area 605; the user's blood pressure data displayed in the second display area 608, the blood pressure data comprising 188 times of blood pressure data monitored at home (abnormal 14 times), 188 times of blood pressure data of diagnostic room monitoring (abnormal 14 times), 188 times of pulse rate data monitored at the clinic area (abnormal 14 times), and 188 times of pulse rate data of manual monitoring (abnormal 14 times) over a period of time; the pulse rate change graph (and medication information data) displayed in the third display area 615; and the medication record displayed in the fourth display area 640.

As shown in FIG. 7, the display interface 700 comprises: the user's basic information displayed in the first display area 710; the user's blood sugar data displayed in the second display area 720, the blood sugar data comprising 188 times of blood sugar data monitored at home (abnormal 14 times), 188 times of blood pressure data of in-hospital monitoring over a period of time; the blood sugar change graph (and medication information data) of in-hospital monitoring displayed in the third display area 730; and the measurement record displayed in the fourth display area 740.

In some embodiments, the first health data comprises a plurality of first data indicating a first detection item (for example, blood pressure), and each of the plurality of first data is associated with one of the at least one user identifier. That is, for each of the plurality of first data, there is a single user identifier associated with at least one user identifier. The second health data comprises second data indicating the first detection item (for example, blood pressure), and step S403 comprises: determining the similarity between the second data and the plurality of first data; and based on the similarity between the second data and the plurality of first data, establishing an association relationship between the second health data and the target user identifier in the at least one user identifier.

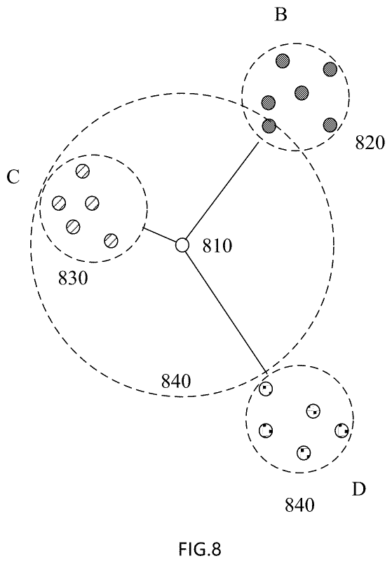

FIG. 8 shows a schematic diagram of second data and a plurality of first data according to an embodiment of the present disclosure. As shown in FIG. 8, the data enclosed by dashed boxes 820, 830, and 840 respectively indicate different sample clusters in the plurality of first data. The sample clusters enclosed by the dashed box 820 indicate that they are associated with the user identifier B, the sample cluster indicated by the dashed frame 830 is associated with the user identifier C, and the sample cluster indicated by the dashed frame 840 is associated with the user identifier D. Determining the similarity between the second data and the plurality of first data comprises: determining the distance values between the second data 810 and each of the plurality of first data respectively to obtain multiple distance values; selecting the first set from the multiple distance values, the first set comprises at least one distance value that meets a predetermined filtering condition (for example, it may be sorted according to the size of the distance value, and several distance values ranked in the top are selected to enter the first set, For example, the distance between the first data and the second data 810 enclosed by the dashed frame 840 falls within the first set); for each (B, C, D) of the at least one user identifier, determining the number of distance values in the first set and associated with each user identifier respectively. As shown in FIG. 8, as indicated by the dashed box 840, the number of distance values in the first set and associated with the user identifier B is 1, the number of distance values in the first set and associated with the user identifier C is 5, and the number of distance values in the first set and associated with the user identifier D is 0.

Based on the similarity between the second data and the plurality of first data, establishing an association relationship between the second health data and the target user identifier in the at least one user identifier comprises: based on the number of distance values associated with each user identifier (that is, the number of distance values in the first set and associated with the user identifier B is 1, the number of distance values in the first set and associated with the user identifier C is 5, and the number of distance values in the first set and associated with the user identifier D is 0), determining the target user identifier; and based on the target user identifier, establishing an association relationship between the second health data and the target user identifier in the at least one user identifier.

For example, based on the number of distance values associated with each user identifier, determining the target user identifier comprises: determining the user identifier C associated with the maximum number of distance values in the first set (i.e., the number of the distance value in the first set and associated with the user identifier C is 5) is the target user identifier.

Alternatively, based on the number of distance values associated with each user identifier, determining the target user identifier comprises: calculating the ratio (i.e. 5/6) of the maximum number of distance values in the first set and associated with each user identifier (i.e., 5 which is the number of the distance value in the first set and associated with the user identifier C) to the sum of the number of distance values in the first set and associated with each user identification (i.e., the sum of 1 which is the number of the distance value in the first set and associated with the user identifier B, 5 which is the number of the distance value in the first set and associated with the user identifier C, and 0 which is the number of the distance value in the first set and associated with the user identifier D), and determining whether the ratio (i.e. 5/6) is greater than the second threshold (for example, it may be set to ⅔); and in response to the ratio being greater than the second threshold (for example, it may be set to ⅔), determining the user identifier associated with the largest distance value in the first set as the target user identifier (C).

In some embodiments, the above-mentioned similarity comparison method may also be combined with step S4031. In this case, FIG. 9 shows at least part of the sub-steps in step S403:

S920, binding different users to the same account of the third terminal 130;

S930, performing measurement by multiple users using the same third terminal 130, in which case, the third terminal 130 needs to determine which user the collected data should be associated with;

S940, calculating the data volume of the health data corresponding to each of the at least one user identifier in the first health data;

S950, determining whether the data volume is greater than a first threshold;

S960, in response to the data volume being greater than the first threshold, performing similarity judgements between the content of the first health data and the content of the second health data, to determine whether there is a target user identifier in the at least one user identifier;

S955, in response to the data volume being not greater than the first threshold, processing the second health data to determine whether there is the target user identifier in the at least one user identifier according to a preset rule.

S965, calculating the ratio of the maximum number of distance values in the first set and associated with each user identifier to the sum of the number of distance values in the first set and associated with each user identifier;

S970, determining whether the ratio is greater than a second threshold;

S980, in response to the ratio being greater than the second threshold, determining that there is a target user identifier in at least one user identifier and determining that the user identifier associated with the largest distance value in the first set is the target user identifier.

S975, in response to the ratio being not greater than the second threshold, determining that there is no target user identifier in the at least one user identifier, in which case, a manual review may be prompted or a new user profile corresponding to the first user may be created in the multi-source health data intelligent archiving system 200.

Through the method provided by the embodiments of the present disclosure, for the new second data, similarity calculation is performed with the plurality of first data, and the user identifier associated with the second data is automatically determined according to the calculation result, thereby realizing the multi-source health data intelligent archiving. The method is accurate and easy to implement, and the reliability may be adjusted by adjusting the second threshold, which improves the efficiency of the system, reduces manual intervention, and improves user experience. In addition, if the second data is similar to the sample clusters of the first data associated with different user identifiers (that is, in the case where the ratio of the maximum number of distance values in the first set and associated with each user identifier to the sum of the number of distance values in the first set and associated with each user identifier is relatively small), in order to ensure the validity of the data, the user may be prompted in the application program, and the user may manually distribute it to the actual user. In this way, in response to the judgement rule, it may be determined that the second data should not belong to the currently existing user identifier and be eliminated, or prompt the user for manual confirmation.

In some embodiments, the second data has a different data format from the plurality of first data. Before step S4031, the data processing method further comprises: converting the format of the second data into the same format as the format of the plurality of first data. For example, the big data intelligent analysis subsystem 200 may be configured to perform data pre-processing, and the process of performing the pre-processing may comprise, for example, operations such as data cleaning, missing value recognition, missing value processing, standardization, and normalization. In this way, the health data from different database systems may have the same format and facilitate subsequent operations such as intelligent archiving and user data trend analysis.

In some embodiments, the first health data comprises a plurality of first data indicating a first detection item, and each of the plurality of first data is associated with one of the at least one user identifier, as shown in FIG. 10a, the second health data comprises second data indicating the first detection item. Based on the first health data and the second health data, establishing an association relationship between the second health data and the target user identifier in the at least one user identifier comprises: based on the second data 1010 and a first prediction model 1020, obtaining a prediction result 1030, the first prediction model 1020 being trained based on the association relationship between each of the plurality of first data and at least one user identifier; and based on the prediction result 1030, establishing an association relationship between the second health data and the target user identifier in the at least one user identifier. For example, based on the second data and the first prediction model, obtaining the prediction result comprises: combining the second data with data of other dimensions associated with the second data to form a data sample; normalizing the data sample; and inputting the unified data sample to the first prediction model to obtain the prediction result. For example, the first predictive model is a neural network model.

Some embodiments are described below by taking the first prediction model being a multi-layer classification neural network model as an example.

More dimensional data characteristics of users may be integrated, such as other health data measured, user basic information, user behaviour habits, etc., to form a multi-dimensional feature set, and then normalize it as a feature set to construct training samples. A multi-layer classification neural network may be constructed, and the training samples may be used for supervised learning to train the multi-layer classification neural network.

For example, when there are three user identifiers U1, U2, U3 in the system, take the blood pressure data xᵢ (i.e., the second data) measured last time being associated with one of the three user identifiers U1, U2, U3 as an example, explain the process of multi-layer classification neural network training and classification operation.

First, construct training samples. The training samples are multi-dimensional data, and the multi-dimensional data is constructed as follows:

Basic user information: height x₁, age x₂, gender x₃ (male 0, female 1); user lifestyle: smoking x₄ (yes 1, no 0), drinking x₅ (yes 1, no 0), living environment x₆ (southern cities 0, southern rural 1, northern city 2, northern rural 3), labor level x₇ (bed rest 0, light manual labor 1, moderate manual labor 2, heavy manual labor 3), exercise status x₈ (substantially no exercise 0, an average of 1 time a week on average 1, 2 times a week on average 2, and 3 times on average 3 . . . ) etc.; the user's latest health monitoring data: blood pressure x₉, blood sugar x₁₀, sleep duration x₁₁, body fat x₁₂ . . . ; and other possible interactions with the user condition factors related to health data (such as measurement time x₁₃, medication status x₁₄, etc.), up to xₙ.

the above data is normalized to form training samples comprising multi-dimensional data. In order to enrich the sample size of training samples, relevant data at different moments in history may be taken to construct training samples to train a multi-layer classification neural network. It should be understood that, in order to improve the accuracy of the multi-layer classification neural network, it is also possible to select only the health data in the most recent period of time as the training samples. The data set may be, for example, as shown in Table 1 below (where t1-tn are different moments, and U1, U2, and U3 are different user identifiers):

TABLE 1

| | | The user identifier |
|---|---|---|
| | | associated with the |
| | | training samples (i.e., |
| | | the result label for |
| moment | Training samples | supervised learning) |
| t1 | [x₁^{t1u1}, x₂^{t1u1}, x₃^{t1u1} . . . xₙ^{t1u1}] | U1 |
| | [x₁^{t1u2}, x₂^{t1u2}, x₃^{t1u2} . . . xₙ^{t1u2}] | U2 |
| | [x₁^{t1u3}, x₂^{t1u3}, x₃^{t1u3} . . . xₙ^{t1u3}] | U3 |
| t2 | [x₁^{t2u1}, x₂^{t2u1}, x₃^{t2u1} . . . xₙ^{t2u1}] | U1 |
| | [x₁^{t2u2}, x₂^{t2u2}, x₃^{t2u2} . . . xₙ^{t2u2}] | U2 |
| | [x₁^{t2u3}, x₂^{t2u3}, x₃^{t2u3} . . . xₙ^{t2u3}] | U3 |
| t3 | [x₁^{t3u1}, x₂^{t3u1}, x₃^{t3u1} . . . xₙ^{t3u1}] | U1 |
| | [x₁^{t3u2}, x₂^{t3u2}, x₃^{t3u2} . . . xₙ^{t3u2}] | U2 |
| | [x₁^{t3u3}, x₂^{t3u3}, x₃^{t3u3} . . . xₙ^{t3u3}] | U3 |
| . . . | . . . | . . . |
| tn | [x₁^{tnu1}, x₂^{tnu1}, x₃^{tnu1} . . . xₙ^{tnu1}] | U1 |
| | [x₁^{tnu2}, x₂^{tnu2}, x₃^{tnu2} . . . xₙ^{tnu2}] | U2 |
| | [x₁^{tnu3}, x₂^{tnu3}, x₃^{tnu3} . . . xₙ^{tnu3}] | U3 |

When using the trained multi-layer classification neural network to classify the second data xᵢ, combine the second data xᵢ with the data of other dimensions associated with the second data [x₁, x₂, x₃, x₄ . . . xᵢ₋₁, xᵢ₊₁ . . . xₙ] (the data of other dimensions [x₁, x₂, x₃, x₄ . . . xᵢ₋₁, xᵢ₊₁ . . . xₙ] may be measured simultaneously with xᵢ, or the basic data of the most recent measurement may be taken. The closer the time is close to xᵢ, the more accurate the prediction result will be) to form the data sample Xᵢ=[x₁, x₂, x₃, x₄ . . . xᵢ . . . xₙ], normalize the data sample Xᵢ and input it to the trained neural network for prediction to get the prediction result 1030. For example, the prediction result may be: indicating that it is associated with the user identifier U1 in the case of 0≤the prediction result<⅓, indicating that it is associated with the user identifier U2 in the case of ⅓≤the prediction result<⅔, and indicating that it is associated with the user identifier U3 in the case of ⅔≤prediction result<1. According to the prediction result 1030, the second data xᵢ to be archived may be associated with one of the three user identifiers U1, U2, U3, that is, the second data xᵢ may be intelligently archived to a user's profile.

In this way, the use of multi-layer classification neural network to realize the intelligent archiving of the second health data improves the sensitivity of the finally obtained first prediction model to the second health data, so that the first prediction model may be used to accurately select the target user identifier associated with the second detection data. In addition, the multi-layer classification neural network may be used to further solve the problems of other methods being insensitive to abnormal data and similarities of multiple users being close.

In some embodiments, similar training samples may be used to train a single-class neural network, and the output of the single-class neural network is “Yes (that is, the second data is associated with the user identifier used to train the single-class neural network)” or “No (that is, the second data is not associated with the user identifier used to train the single-class neural network)”. For example, there are 3 user identifiers in the current system: U1, U2, U3. When the data volume is sufficient, the single-class neural network 1022 is trained using multiple training samples corresponding to the user identifier U1, the single-class neural network 1024 is trained using multiple training samples corresponding to the user identifier U2, and the single-class neural network 1026 is trained using multiple training samples corresponding to the user identifier U3. The three single-class neural networks 1022, 1024, and 1026 are respectively used to determine whether the second data 1010 is associated with the user identifier U1, U2, or U3 (each single-class neural network may only correspond to one user identifier). As shown in FIG. 10b, for the second data 1010 newly obtained by the third computing device 120, when determining whether there is a target user identifier among the three user identifiers U1, U2, U3, the second data 1010 may be input to the three single-class neural networks, to obtain the output results 1032, 1034, and 1036 of the three single-class neural networks, respectively: “Yes (that is, the second data is associated with the user identifier used to train the neural network)” or “No (that is, the second data is not associated with the user identifier used to train the neural network)”. In this way, if the output results 1032, 1034, and 1036 of the three single-class neural networks are all “No”, it may be determined that there is no target user identifier does in the 3 user identifiers, and manual review may be prompted for archiving and the abnormal health data is eliminated or a new profile of abnormal health data is established. If there is a single “Yes” in the output results 1032, 1034, and 1036 of the three single-class neural networks, it may be determined that there is target user identifier in the three user identifiers, and the user identifier corresponding to the single-class neural network that outputs the result of “Yes” is the target user identifier. If there are two or more “Yes” in the output results 1032, 1034, and 1036 of the three single-class neural networks, a manual review needs to be prompted. In this way, it is possible to base on the content of the first health data and the content of the second health data, determine whether there is a target user identifier in the at least one user identifier (see the description of step S4031 above).

In some embodiments, referring back to FIGS. 6-7, based on the health data sequence, analysing the health status of the user associated with the target user identifier comprises: obtaining a user feature sequence associated with the target user identifier; based on the health data sequence, the user feature sequence, and a second prediction model, obtaining an analysis result of the user's health status associated with the target user identifier, wherein the second prediction model is trained based on the user's historical health data sequence, historical user feature sequence and historical health status.

For example, after associating the health data belonging to the user A1 obtained by each database system with the A1 user, the third computing device 120 may, for example, obtain the blood pressure data sequence, the blood sugar data sequence and the user characteristics sequence of the user A1 corresponding to the user A1 in the most recent period of time. The user characteristics sequence of the user A1 may comprise basic information provided by the user through the second terminal 130. The third computing device 120 may combine the blood pressure data sequence, the blood sugar data sequence, and user feature sequence of user A1 to obtain the data sequence to be analysed; input the data sequence to be analysed into the second prediction model to obtain the health status analysis result corresponding to user A1, which is convenient for the user A1, the doctor or the health manager to view the analysis results of the health status of the user A1. If the monitoring status analysis result is abnormal, the health status analysis result and user A1's drug prescription data stored in the data lake are sent to the second terminal 130 of user A1, and the health status analysis result and user A1's drug prescription data is sent to the second computing device 112 associated with the second data system, which is convenient for doctors or health managers to return visits to the user A1 in time, and may remind the health managers or patients to take corresponding measures and control in time. For example, the second prediction model may be at least one of an ARIMA model, a neural network model, or a Prophet model.

For example, the third computing device 120 may arrange the blood pressure data from different sources of the user's home, diagnostic room, clinic area, and manual monitoring according to time nodes to form a health data sequence; combine the user's age, height, gender, living habits and other information into a user feature sequence; predict the data trend based on the health data sequence, the user feature sequence and the second prediction model. If the blood pressure data is predicted to have an upward trend, the multi-source health data intelligent archiving system 200 may remind the management user (doctor/health manager) on the management side to return visit to the patient, and at the same time, the patient may be reminded on the mini program end to remind the patient to pay attention to diet, exercise, medication or timely medical treatment, etc. The same method may be used for blood sugar or other data.

For example, the third computing device 120 may arrange the blood pressure data from different sources of users' homes, diagnostic rooms, clinic areas, and manuals according to time nodes to form a health data sequence Xₜ₁; arrange blood sugar data Xₜ₂ in a similar manner; and possibly more other health data sequences X_{t3 . . . tn}; combine the user's age, height, gender, living habits and other information into a feature sequence X_{feature}; based on multiple health data sequences Xₜ₁, Xₜ₂, X_{t3 . . . tn}, user feature sequences and the second predictive models, predict the overall health trend of patients. If there is an abnormality in the prediction of the health trend, the multi-source health data intelligent archiving system 200 may remind the management end user (doctor/health manager) on the management side to return visit to the patient, and at the same time, the patient may be reminded on the mini program end to remind the patient to pay attention to diet, exercise, medication or timely medical treatment, etc. In this way, it may also solve the problem that many elderly people do not know how to use smart phones. The multi-source health data intelligent archiving system 200 automatically tracks and analyses the user's health status.

The data processing method provided by the embodiments of the present application may obtain the user's health status analysis result associated with the target user identifier based on data from different database systems; achieve high-precision archiving of the user's health data, and ensure the obtained quality of the user health data and the integration of multi-source health data comprising archived health data, and the analysis of the user's health status based on the integration data, facilitating users to intuitively, timely and comprehensively grasp their own health status. In this way, using big data analysis technology, with comprehensive analysis of personal multi-source data and early warning analysis of residents' health, when there are abnormal early warnings, residents may take timely response measures, such as timely medical treatment.

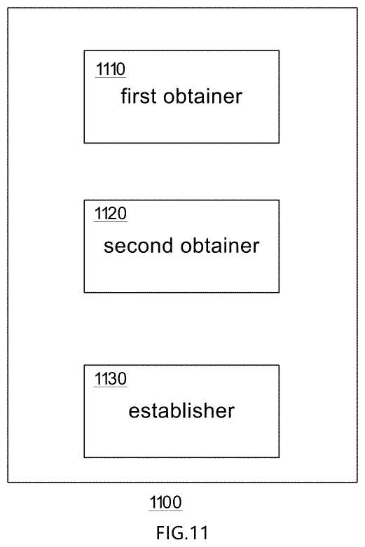

As shown in FIG. 11, an embodiment of the present disclosure also provides a data processing device 1100, the data processing device 1100 comprising: a first obtainer configured to obtain first health data, the first health data marked as being associated with at least one user identifier; a second obtainer configured to obtain second health data, the second health data comprises health data of the first user; and a establisher configured to establish an association relationship between the second health data and a target user identifier in the at least one user identifier based on the first health data and the second health data, wherein the target user identifier is associated with the first user.

The data processing device may have advantages and effects similar to the above-mentioned data processing method, which will not be repeated here.

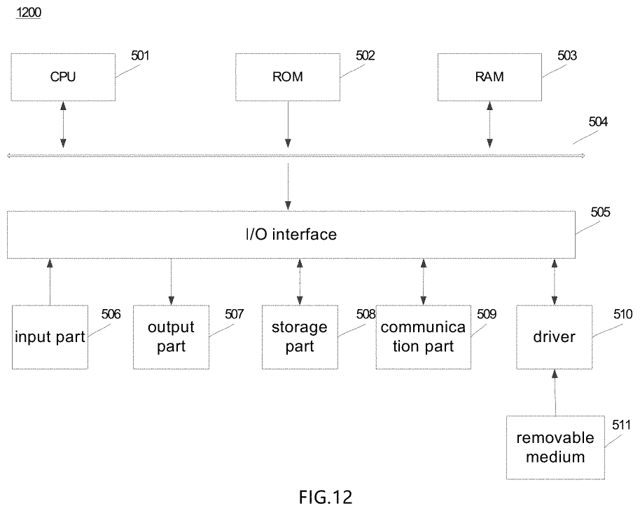

FIG. 12 shows a computing device 1200 according to an exemplary embodiment. The computing device 1200 may be, for example, one of the first computing device 110, the second computing device 112, the third computing device 130, the first terminal 1101, and the second terminal 130. The computing device 1200 may comprise a memory, a processor, and computer instructions stored in the memory and executable on the processor, the processor being configured to realize the data processing method as above when the computer instruction is executed. For example, the storage comprises an IoT data lake.

For example, the computing device 1200 comprises a central processing unit (CPU) 501, which may preform various appropriate actions and processing according to a program stored in a read-only memory (ROM) 502 or a program loaded from a storage part into a random access memory (RAM) 503. In RAM 503, various programs and data required for system operation are also stored. The CPU 501, ROM 502, and RAM 503 are connected to each other via a bus 504. An input/output (I/O) interface 505 is also connected to the bus 504.

The following components are connected to the I/O interface 505: an input part 506 comprising a keyboard, a mouse, etc.; an output part comprising a cathode ray tube (CRT), a liquid crystal display (LCD), etc., and a speaker; a storage part 508 comprising a hard disk, etc.; and the communication part 509 comprising a network interface card such as a LAN card, a modem, and the like. The communication part 509 performs communication processing via a network such as the Internet. The driver is also connected to the I/O interface 505 as needed. A removable medium 511, such as a magnetic disk, an optical disk, a magneto-optical disk, a semiconductor memory, etc., is installed on the drive 510 as needed, so that the computer program read therefrom is installed into the storage part 508 as needed.

In particular, according to an embodiment of the present disclosure, the method described above with reference to the flowchart may be implemented as a computer software program. For example, various embodiments of the present disclosure provide a computer program product, which comprises a computer program carried on a computer-readable medium, and the computer program comprises program code configured to carry out the method shown in the flowchart. In such an embodiment, the computer program may be downloaded and installed from the network through the communication part, and/or installed from a removable medium. When the computer program is executed by the central processing unit (CPU) 501, the above-mentioned functions defined in the system of the present disclosure are executed.

The embodiments of the present disclosure also provide a non-transitory computer-readable storage medium having computer instructions stored thereon, the computer instructions being configured to implement any of the above message processing methods. It should be noted that the non-transitory computer-readable medium shown in the present disclosure may be a computer-readable signal medium or a computer-readable storage medium, or any combination thereof. The computer-readable storage medium may be, for example, but not limited to, an electrical, magnetic, optical, electromagnetic, infrared, or semiconductor system, apparatus or device, or a combination of any of the above. More specific examples of computer-readable storage media may comprise, but are not limited to: electrical connections with one or more wires, portable computer disks, hard disks, random access memory (RAM), read-only memory (ROM), erasable removable Programmable read-only memory (EPROM or flash memory), optical fibre, portable compact disk read-only memory (CD-ROM), optical storage device, magnetic storage device, or any suitable combination of the above. In the present disclosure, a computer-readable storage medium may be any tangible medium that contains or stores a program, and the program may be used by or in combination with an instruction execution system, apparatus, or device. In the present disclosure, a computer-readable signal medium may comprise a data signal propagated in a baseband or as a part of a carrier wave, and a computer-readable program code is carried therein. This propagated data signal may take many forms, comprising but not limited to electromagnetic signals, optical signals, or any suitable combination of the above. The computer-readable signal medium may also be any computer-readable medium other than the computer-readable storage medium. The computer-readable medium may send, propagate, or transmit a program configured to be used by or in combination with an instruction execution system, apparatus or device. The program code contained on the computer-readable medium may be transmitted by any suitable medium, comprising but not limited to: wireless, wire, optical cable, RF, etc., or any suitable combination of the above.

The non-transitory computer-readable storage medium may be comprised in the electronic device described in the embodiments; or it may exist alone without being assembled into the electronic device. The non-transitory computer-readable storage medium stores one or more programs, and the foregoing programs are used by one or more processors for preforming the message processing method described in the present disclosure.

The flowcharts and block diagrams in the drawings illustrate the possible implementation architecture, functions, and operations of the methods, devices, and computer program products according to various embodiments of the present disclosure. In this regard, each block in the flowchart or block diagram may represent a module, program segment, or part of the code, and the above-mentioned module, program segment, or part of the code contains one or more executable instructions configured to realize the specified logical function. It should also be noted that, in some alternative implementations, the functions marked in the block may also occur in a different order from the order marked in the drawings. For example, two blocks shown in succession may actually be executed substantially in parallel, and they may sometimes be executed in the reverse order, depending on the functions involved. It should also be noted that each block in the block diagram or flowchart, and the combination of blocks in the block diagram or flowchart, may be implemented by a dedicated hardware-based system that performs the specified function or operation, or may be implemented by a combination of dedicated hardware and computer instructions.

The units described in the embodiments of the present disclosure may be implemented in either software or hardware, and the described units may also be provided in a processor. The names of these units do not constitute a limitation on the unit itself under certain circumstances. The described unit or module may also be provided in the processor, for example, it may be described as: a processor comprises a first obtainer, a second obtainer, and an establisher. The names of these units or modules do not constitute a limitation on the unit or module itself under certain circumstances. For example, the first obtainer may also be described as “an obtainer configured to obtain the first health data, the first health data marked as being associated with at least one user ID”.

The above description is only a preferred embodiment of the present disclosure and an explanation of the applied technical principles. Those skilled in the art should understand that the scope of the invention involved in this disclosure is not limited to the technical solutions formed by the specific combination of the above technical features, and should also encompass other technical solutions formed by any combination of the above technical features or their equivalent features without departing from the inventive concept, for example, the technical solutions formed by mutual substitution of the above features and the technical features with similar functions disclosed in the present disclosure (but not limited to).

1. A data processing method, the data processing method being carried out by a computing device, the data processing method comprising:
obtaining first health data, the first health data being marked as being associated with at least one user identifier;
obtaining second health data, the second health data comprising health data of a first user; and
based on the first health data and the second health data, establishing an association relationship between the second health data and a target user identifier in the at least one user identifier, wherein the target user identifier is associated with the first user.

2. The data processing method according to claim 1, wherein the second health data and the first health data come from different database systems.

3. The data processing method according to claim 2, wherein based on the first health data and the second health data, establishing an association relationship between the second health data and the target user identifier in the at least one user identifier comprises:
determining whether there is the target user identifier in the at least one user identifier based on the first health data and the second health data; and
in response to presence of the target user identifier in the at least one user identifier, establishing an association relationship between the second health data and the target user identifier.

4. The data processing method according to claim 2, after establishing the association relationship between the second health data and the target user identifier, further comprises:
based on the first health data and the second health data, analysing a health status of a user associated with the target user identifier.

5. The data processing method according to claim 1, wherein the first health data comprises a plurality of first data indicating a first detection item, and each of the plurality of first data is related to one of the at least one user identifier, the second health data comprises second data indicating a first detection item, and based on the first health data and the second health data, establishing an association relationship between the second health data and the target user identifier in the at least one user identifier by performing operations comprising:
determining a similarity between the second data and the plurality of first data; and
based on the similarity between the second data and the plurality of first data, establishing an association relationship between the second health data and the target user identifier in the at least one user identifier.

6. The data processing method according to claim 5, wherein the second data has a data format different from a data format of the plurality of first data, and before determining whether there is the target user identifier in the at least one user identifier based on the first health data and the second health data, the data processing method further comprises:
converting the data format of the second data into a same format as the data format of the plurality of the first data.

7. The data processing method according to claim 3, wherein determining whether there is the target user identifier in the at least one user identifier based on the first health data and the second health data comprises:
calculating a data volume of the health data corresponding to each of the at least one user identifier in the first health data;
determining whether the data volume is greater than a first threshold;
in response to the data volume being greater than the first threshold, determining whether there is the target user identifier in the at least one user identifier based on a content of the first health data and a content of the second health data; and
in response to the data volume being not greater than the first threshold, determining whether there is the target user identifier in the at least one user identifier according to a preset rule.

8. The data processing method according to claim 5, wherein the determining the similarity between the second data and the plurality of first data comprises:
respectively determining a distance value between the second data and each of the plurality of first data to obtain a plurality of distance values;
selecting a first set from the plurality of distance values, wherein the first set comprises at least one distance value that meets a predetermined filtering condition;
for each of the at least one user identifier, respectively determining a number of distance values in the first set and associated with each user identifier,
wherein, based on the similarity between the second data and the plurality of first data, establishing an association relationship between the second health data and the target user identifier in the at least one user identifier by performing operations comprising:
determining the target user identifier based on the number of distance values associated with each user identifier; and
based on the target user identifier, establishing an association relationship between the second health data and the target user identifier in the at least one user identifier.

9. The data processing method according to claim 8, wherein the determining the target user identifier based on the number of distance values associated with each user identifier comprises:
determining the user identifier associated with a largest number of distance values in the first set as the target user identifier.

10. The data processing method according to claim 8, wherein the determining the target user identifier based on the number of distance values associated with each user identifier comprises:
calculating a ratio of a maximum number of distance values in the first set and associated with each user identifier to a sum of the number of distance values in the first set and associated with each user identifier;
determining whether the ratio is greater than a second threshold; and
in response to the ratio being greater than the second threshold, determining the user identifier associated with largest distance values in the first set as the target user identifier.

11. The data processing method according to claim 1, wherein the first health data comprises a plurality of first data indicating a first detection item, and each of the plurality of first data is related to one of the at least one user identifier, the second health data comprises second data indicating a first detection item, and based on the first health data and the second health data, and wherein establishing an association relationship between the second health data and the target user identifier in the at least one user identifier comprises:
obtaining a prediction result based on the second data and a first prediction model, and the first prediction model is trained based on the association relationship between each of the plurality of first data and the at least one user identifier; and
establishing an association relationship between the second health data and the target user identifier in the at least one user identifier based on the prediction result.

12. The data processing method according to claim 4, wherein based on the first health data and the second health data, analysing the health status of the user associated with the target user identifier comprises:
determining a collection moment corresponding to the first health data and a collection moment corresponding to the second health data;
arranging the first health data and the second health data in chronological order to obtain a health data sequence; and
based on the health data sequence, analysing the health status of the user associated with the target user identifier.

13. The method of claim 12, wherein based on the health data sequence, analysing the health status of the user associated with the target user identifier comprises:
obtaining a user feature sequence associated with the target user identifier; and
based on the health data sequence, the user feature sequence, and a second prediction model, obtaining an analysis result of the health status of the user associated with the target user identifier,
wherein the second prediction model is trained based on a user's historical health data sequence, historical user feature sequence and historical health status.

14. The data processing method according to claim 11, wherein the first prediction model is a neural network model.

15. The data processing method according to claim 13, wherein the second prediction model is at least one of an ARIMA model, a neural network model, or a Prophet model.

16. The data processing method according to claim 11, wherein the obtaining a prediction result based on the second data and the first prediction model comprises:
combining the second data with data of other dimensions associated with the second data to form a data sample;
normalizing the data sample; and
inputting the normalized data sample into the first prediction model to obtain a prediction result.

17. A data processing device, comprising:
a first obtainer configured to obtain first health data, the first health data being marked as being associated with at least one user identifier;
a second obtainer configured to obtain second health data, the second health data comprising health data of a first user; and
an establisher configured to establish an association relationship between the second health data and a target user identifier in the at least one user identifier based on the first health data and the second health data, wherein the target user identifier is associated with the first user.

18. A computing device, the computing device comprising a memory, a processor, and computer instructions stored in the memory and executable on the processor, the processor being configured to implement the data processing method according to claim 1 when the computer instructions are executed.

19. The computing device of claim 18, wherein the memory comprises an IoT data lake.

20. A non-transitory computer-readable storage medium having computer instructions stored thereon, the computer instructions being configured to implement the data processing method according to claim 1.
